(19) United States
(12) Patent Application Publication
(10) Pub. No.: US 2018/0256460 A1
(43) Pub. Date: Sep. 13, 2018

(54) STAIN-REMOVING ORAL COMPOSITION

(71) Applicant: LOTTE CO., LTD., TOKYO (JP)

(72) Inventors: Kensuke SAITO, Saitama-shi (JP); Teppei DOI, Saitama-shi (JP); Yoji SAEKI, Saitama-shi (JP); Masahiro TANI, Tokyo (JP); Yoko ITO, Tokyo (JP); Yasutaka HIRAOKA, Saitama-shi (JP); Yuuki NAKAMURA, Saitama-shi (JP)

(21) Appl. No.: 15/978,158

(22) Filed: May 13, 2018

Related U.S. Application Data
(62) Division of application No. 15/113,816, filed on Jul. 22, 2016.

(30) Foreign Application Priority Data
Jan. 24, 2014 (JP) 2014-011676

(51) Int. Cl. A61K 8/24; A61Q 11/00; A23G 4/06; A61K 8/362; A61K 8/34; A61K 8/02; A61K 8/365

(52) U.S. Cl. CPC A61K 8/24; A61Q 11/00; A23G 4/06; A61K 8/365; A61K 8/362; A61K 8/345; A61K 8/0204; A23V 2002/00

(57) ABSTRACT

To increase the esthetic property by removing stains deposited on the dental surface, there is provided a stain-removing oral composition containing 0.33% by weight to 2.0% by weight of sodium metaphosphate.

### TECHNICAL FIELD

The invention of the present application relates to a food product such as chewing gum which has a stain-removing effect.

### BACKGROUND ART

Anterior teeth are decisive in the esthetic property of a human's face, and play a quite important role as a distinctive existence having a different color from the gingival color, the lip color or the like. That is, teeth are necessarily observed to the other person in face-to-face communication; if the teeth are white and clean, the conversation will be pleasant. Thus, the impression of a person is greatly affected by the color of his/her teeth, apart from oral hygiene viewpoints.

Discoloration on the tooth surface is said to result from deposition of food-derived pigments (such as tea, coffee and red wine) as extrinsic stains, discoloration due to Maillard reaction by denaturation of glycoproteins in the saliva covering the dental surface, discoloration by sulfur-containing amino acids or metals, and discoloration of double bond moieties in proteins.

In recent years, with the rising consciousness of “make teeth white and clean,” in-office whitening using hydrogen peroxide and at-home whitening using urea peroxide are conducted. As dentifrices or the like, those with an ingredient which whitens teeth have become commercially available.

However, while in-office whitening has a very high bleaching effect, there is a concern that the use of a high concentration of 30% to 35% of hydrogen peroxide can damage the teeth. Also in at-home whitening, it takes several hours for urea peroxide to decompose and effectively function, putting a restriction on everyday life, which is thus problematic. Moreover, an attempt has been made to remove ingredients discoloring the teeth by incorporating a polyphosphate into chewing gum (PTL 1). However, there still remains a problem to be solved in its whitening effect.

The present inventors aim at developing a food product capable of removing extrinsic staining of teeth safely and tastily at any time at a low price. When chewing gum which is a food product is used as a base material, materials which can be used are limited to abrasive agents or chemical agents which must be designated as food ingredients or food additives. Then, the present inventors focused on sodium metaphosphate, upon which stain removal or stain prevention study in dentifrices has been made heretofore (NPL 1). Sodium metaphosphate is designated as a food additive in Japan, and there is a report of a clinical study on the stain-removing effect of chewing gum containing sodium metaphosphate overseas. In the present invention, as an active ingredient, sodium metaphosphate is taken as a candidate for the stain-removing material, and the effects of sugar alcohols and acidulants, which are ingredients of chewing gum, are evaluated, while the stain-removing effect is investigated in an extract solution of chewing gum product by the saliva.

### CITATION LIST

### Patent Literature

PTL 1: Japanese Patent Application Laid-Open No. 2006-6264

### Non Patent Literature

NPL 1: J Clin Dent. 13: 15-8, 2002

### SUMMARY OF INVENTION

### Technical Problem

In order to increase the esthetic property by removing stains deposited on the dental surface, various methods have been studied such as polishing of the dental surface with an abrasive agent, proteolysis by enzymes, or ion exchange by phosphates, and a number of oral compositions have been proposed. However, most of the actual range of application is medicated dentifrices, which are limited to tooth brushing. Thus, the present inventors aimed at the development of a food product such as chewing gum with a stain-removing effect which can be conveniently consumed.

### Solution to Problem

To enable stain removal by consumption of a food product, there is provided a chewing gum containing 0.33% by weight to 2.0% by weight of sodium metaphosphate, which is used as a food additive.

### Advantageous Effects of Invention

There is a growing interest in the field of esthetic dentistry, and it is believed that handy whitening by consumption of chewing gum will lead to opening up of a new market.

In recent years, with rising consciousness of “make teeth white,” attention has been paid to the field of esthetic dentistry centering on whitening. The present inventors conducted diligent research, and as a result, found that chewing gum containing sodium metaphosphate has an effect of removing stains derived from food, such as coffee, to whiten the teeth.

The invention of the present application relates to an oral composition whereby removal of food-derived stains and “whitening of teeth” can be handily practiced, and the development of various chewing gum will be expected.

### BRIEF DESCRIPTION OF DRAWINGS

FIG. 1 is a graph showing an evaluation result of the stain-removing effect of sodium metaphosphate.

FIG. 2A is a graph showing the evaluation result of the stain-removing effect of sugar alcohols themselves.

FIG. 2B is a graph showing the influence of the sugar alcohols on the stain-removing effect of sodium metaphosphate.

FIG. 3A is a graph showing the evaluation result of the stain-removing effect of acidulants themselves.

FIG. 3B is a graph showing the influence of the acidulants on the stain-removing effect of sodium metaphosphate.

FIG. 3C is a graph showing the influence of a change in pH on the stain-removing effect of sodium metaphosphate.

FIG. 3D is a graph showing the influence of organic acid anion moiety on the stain-removing effect of sodium metaphosphate.

FIG. 4A is a graph showing an evaluation result of the stain-removing effect of chewing gum extract solutions.

FIG. 4B is an image showing hydroxyapatite discs of the groups after 49 times of treatment and before and after staining: from the left hand, the disc immediately after staining, the group treated with the saliva alone, the group treated with control gum, the group treated with 0.33% sodium metaphosphate-containing gum, the group treated with 1.0% sodium metaphosphate-containing gum and the unstained disc.

### DESCRIPTION OF EMBODIMENTS

The present invention was made by studying the application of sodium metaphosphate with dental stain-removing effect to chewing gum.

The invention of the present application relates to a stain-removing oral composition containing sodium metaphosphate.

The invention of the present application relates to the stain-removing oral composition as described above further containing a sugar alcohol.

The invention of the present application relates to the stain-removing oral composition as described above further containing an acidulant.

The invention of the present application relates to the stain-removing oral composition as described above wherein the sugar alcohol is maltitol or xylitol.

The invention of the present application relates to the stain-removing oral composition as described above wherein the acidulant is selected from citric acid, malic acid and fumaric acid.

The invention of the present application relates to the stain-removing oral composition as described above wherein a content of the sodium metaphosphate is 0.33% by weight to 2.0% by weight.

First, to examine the effect of sodium metaphosphate, a hydroxyapatite disc formed thereon a pellicle as a protective film of the saliva—an acquired film formed of a protein contained in the saliva—was stained with staining liquid. The disc was immersed in a saliva solution to which sodium metaphosphate had been added, and the color difference of the disc surface was evaluated. As a result, a significant decrease in the color difference was observed.

Next, the influence of sugar alcohols and acidulants contained in chewing gum on stain removal was examined. As a result, sugar alcohols had no influence, but acidulants showed the significant inhibition.

Moreover, an extract solution by the saliva of sodium metaphosphate-containing chewing gum designed so that the saliva pH would not become 6 or lower even though an acidulant was added showed a significant decrease in the color difference compared to an extract solution of control gum. With continuous consumption of sodium metaphosphate-containing chewing gum 7 times daily for 1 week, a stain-removing effect would be expected.

Examples of the present invention and Testing Examples will be described below, but the scope of the present invention is not limited by these.

### Example 1

In the case of incorporating sodium metaphosphate in a chewing gum, acridity or irritation originated from sodium metaphosphate can stand out, breaking the balance of flavor, in some cases. For this reason, an attempt was made to reduce the acridity or irritation originated from sodium metaphosphate by adjusting the carbohydrate ingredient which is contained in chewing gum in the largest amount.

### (Testing Method (1))

TABLE 1
Gum formulation for sensory testing (1)

| Ingredient | Content (% by weight) |
|---|---|
| Gum base | 16 |
| *Carbohydrate ingredient | 80 |
| Flavoring | 1.4 |
| Softener/thickener | 0.6 |
| Sweetener with high sweetness | 0.9 |
| Acidulant | 0.77 |
| Sodium metaphosphate | 0.33 |
| Total | 100 |

*The carbohydrate ingredient was one selected from xylitol, maltitol, mannitol, erythritol and sugar.

On the basis of Table 1, while the content of sodium metaphosphate was fixed at 0.33% by weight, one carbohydrate ingredient was selected from xylitol, maltitol, mannitol, erythritol and sugar, then 80% by weight thereof was incorporated. Thus, testing was conducted on the influence of the difference of the carbohydrate ingredient on the flavor of chewing gum. The method of producing gum follows a conventional method, and 4 expert panelists tried and evaluated the produced gum.

Evaluation items were 4 items: “Intensity of acridity,” “Irritation,” “Quality of sweetness,” and “Comprehensive evaluation” considering these 3 items comprehensively. With respect to the rating score being the evaluation criteria, the following was applied. For “Intensity of acridity” and “Irritation,” 5: not unpleasant at all, 4: not unpleasant, 3: slightly unpleasant, but to an extent that general consumers will not find it unpleasant, 2: slightly unpleasant, and 1: unpleasant. For “Quality of sweetness” and “Comprehensive evaluation,” 5: very good, 4: good, 3: fair (to an extent that general consumers will find it taste good), 2: slightly poor, 1: poor. It was noted that with respect to “Quality of sweetness,” evaluation was made in regard not only to the carbohydrate ingredient used in chewing gum to be evaluated but also to the gum as a whole including sodium metaphosphate, etc., considering mutual influences between ingredients. On the basis of the above criteria, the rating scores were in 0.5 steps between 1 and 5, and the evaluation was made on 9-scale.

### (Testing Results)

The results for sensory evaluation of gum when the carbohydrate ingredient in chewing gum containing 0.33% by weight of sodium metaphosphate was changed are shown in Table 2.

TABLE 2
The influence of altering the kind of carbohydrate ingredient on the
flavor of gum

| Carbohydrate | Intensity | | Quality | | |
|---|---|---|---|---|---|
| ingredient | of | | of | Comprehensive | |
| used | acridity | Irritation | sweetness | evaluation | Remarks |
| Xylitol | 4.8 | 5.0 | 5.0 | 5.0 | Sweetness |
| | | | | | with body, |
| | | | | | and |
| | | | | | negligible |
| | | | | | acridity |
| Erythritol | 2.3 | 2.8 | 2.3 | 2.5 | Poor |
| | | | | | dissolution |
| | | | | | of |
| | | | | | sweetness, |
| | | | | | intense |
| | | | | | irritation |
| | | | | | feeling |
| Maltitol | 3.0 | 3.5 | 3.1 | 2.9 | Balanced |
| | | | | | sweetness |
| Mannitol | 2.0 | 2.3 | 1.8 | 1.9 | Acridity |
| | | | | | appears |
| | | | | | intensely |
| Sugar | 2.3 | 2.3 | 2.8 | 2.5 | Similar |
| | | | | | trend as |
| | | | | | using |
| | | | | | mannitol |

As observed in Table 2, when xylitol was used as the carbohydrate ingredient, acridity and irritation originated from sodium metaphosphate were best masked, and sweetness with good quality appeared. Then, when maltitol was used as the carbohydrate ingredient, while the comprehensive evaluation was not so high, unpleasant feeling from the acridity and irritation originated from sodium metaphosphate was scarcely gained, and well-balanced sweetness appeared, as could be observed. It was noted that when mannitol, erythritol or sugar was used as the carbohydrate ingredient, there was a tendency of feeling acridity and irritation unpleasant.

It has been revealed from these results that xylitol or maltitol is suitably used as the carbohydrate ingredient in the sodium metaphosphate-containing chewing gum.

### Example 2

### (Testing Method (2))

TABLE 3
Gum formulation for sensory testing (2)

| Ingredient | Content (% by weight) |
|---|---|
| Gum base | 16 |
| *Xylitol | 0-80 |
| *Maltitol | 80-0 |
| Flavoring | 1.4 |
| Softener/thickener | 0.6 |
| Sweetener with high sweetness | 0.9 |
| Acidulant | 0.77 |
| *Sodium metaphosphate | 0.33-2.5 |
| Total | 100 |

*Xylitol and maltitol were used in combination as the carbohydrate ingredients, and each content was adjusted as appropriate depending on the amount of sodium metaphosphate blended.

On the basis of Table 3, the content of sodium metaphosphate was varied between 0.33 and 2.5% by weight, and xylitol and maltitol were blended as carbohydrate ingredients. Thus, testing was conducted on the influence of the difference in the sodium metaphosphate content, and the difference in the xylitol content on the flavor of chewing gum.

It was to be noted that when the sodium metaphosphate content was 0.33% by weight, the total amount of xylitol and maltitol was adjusted to be 80% by weight. When the sodium metaphosphate content was 0.80% by weight, the total amount of xylitol and maltitol were adjusted to be 79.53% by weight. When the sodium metaphosphate content was 2.0% by weight, the total amount of xylitol and maltitol was adjusted to be 78.33% by weight. When the sodium metaphosphate content was 2.5%, the total amount of xylitol and maltitol was adjusted to be 77.83% by weight.

The content of xylitol was 0% by weight, 10% by weight, 16% by weight, 64% by weight, or about 80% by weight based on the total amount of chewing gum. When the xylitol content was 0 to 64% by weight, the amount of maltitol blended was adjusted so that the total of xylitol and maltitol be the specified amount. When the carbohydrate ingredient used was totally xylitol, that is, when maltitol was not used, when the sodium metaphosphate content was 0.33% by weight, 0.80% by weight, 2.0% by weight, or 2.5% by weight, the xylitol content was adjusted to be 80% by weight, 79.53% by weight, 78.33% by weight, or 77.83% by weight, respectively.

The method of producing gum follows a conventional method, and 4 expert panelists tried and evaluated the produced gum.

Evaluation item was 1 item: “Comprehensive evaluation” considering “Intensity of acridity,” “Irritation,” and “Quality of sweetness” comprehensively. With respect to the rating scores being the evaluation criteria, 5: very good, 4: good, 3: fair (to an extent that general consumers will find it taste good), 2: slightly poor, 1: poor. On the basis of the above criteria, the rating scores were in 0.5 steps between 1 and 5, and the evaluation was made on 9-scale.

### (Testing Results)

The results for sensory evaluation of gum when the content of sodium metaphosphate and the content of xylitol were varied are shown in Table 4.

TABLE 4
The influence of varying the contents of xylitol and sodium
metaphosphate on the flavor of gum

| | | | | |
|---|---|---|---|---|
| Sodium metaphosphate content | 0.33 | 0.80 | 2.0 | 2.5 |
| (% by weight) | | | | |
| Xylitol content 0% by weight | 2.9 | 2.9 | 2.1 | 1.7 |
| Xylitol content 10% by weight | 3.3 | 3.1 | 3.0 | 1.9 |
| Xylitol content 16% by weight | 4.0 | 3.7 | 3.1 | 2.1 |
| Xylitol content 64% by weight | 4.7 | 4.4 | 3.2 | 2.5 |
| Xylitol content about 80% by weight | 5.0 | 4.6 | 3.6 | 2.9 |
| Remarks | *1 | *2 | *3 | *4 |

*1 Irritation and acridity appeared, but were significantly improved by incorporating xylitol, and the balance between them and the flavor was gained
*2 Acridity was reduced and the quality of sweetness improved by incorporation of xylitol
*3 Even though xylitol was incorporated, irritation and acridity somewhat appeared, but to an extent that it did not feel unpleasant
*4 There was a grinding feeling and irritation from the beginning of chewing, and even though the xylitol content was about 80% by weight, strong acridity appeared

As observed in Table 4, the incorporation of xylitol generally reduced unpleasant feeling from acridity or irritation originated from sodium metaphosphate, and improved the quality of sweetness. However, as the content of sodium metaphosphate increased, the evaluation result worsened, and a tendency was observed that unpleasant feeling from acridity or irritation occurred. Meanwhile, when the xylitol content was 10 to 80% by weight, even though 2.0% by weight of sodium metaphosphate was incorporated, the following evaluation results were obtained: the rating scores were 3.0 or more, i.e., satisfied an extent that general consumers would find it taste good. However, when 2.5% by weight of sodium metaphosphate was incorporated, even though the carbohydrate ingredient was totally xylitol, i.e., 77.83% by weight of xylitol was incorporated, the evaluation result was as follows: the rating score was less than 3.0, i.e., did not satisfy an extent that general consumers would find it taste good. When the xylitol content was 0% by weight, i.e., xylitol was not used, even though the content of sodium metaphosphate was as small as 0.33% by weight, the evaluation result was not in the acceptable range.

Based on these results, it was observed that in the sodium metaphosphate-containing chewing gum, when the content of sodium metaphosphate was 2.0% by weight or less, and the xylitol content was 10 to 80% by weight, chewing gum was obtained with good sweetness which did not give unpleasant feeling from acridity or irritation originated from sodium metaphosphate.

### Example 3

Based on the results of Examples 1 and 2 above, the stain-removing effects of the sodium metaphosphate-containing chewing gum were examined by the following method.

### 1. Sample

### 1) Material Used

### (1) Sodium Metaphosphate (Manufactured by Taihei Chemical Industrial Co., Ltd.)

### (2) Sugar Alcohols

Xylitol
Maltitol

### (3) Acidulants

Citric acid
Malic acid
Fumaric acid
   2) Sample Chewing Gum (Tablet Gum 1.5 g/Tablet)

Three kinds of chewing gum were used in this Example, and each composition is shown in Table 5. That is, control gum not containing sodium metaphosphate, and two kinds of sodium metaphosphate-containing gum respectively containing 0.33% by weight and 1.0% by weight of sodium metaphosphate were used.

TABLE 5
Composition of sample chewing gum (% by weight)

| | | Sodium metaphosphate- |
|---|---|---|
| | Control gum | containing gum |
| Sugar alcohol | | |
| Xylitol | 17 | 17 |
| Maltitol | 60 | 60 |
| Acidulant (citric acid/malic | 0.5 | 0.5 |
| acid/fumaric acid) | | |
| Sodium metaphosphate | — | 0.33 or 1.0 |

### 2. Collection and Processing of Saliva

Each research volunteer was asked to masticate one tablet of salivary gum (Morita Corporation), and about 30 ml of the saliva was collected respectively. The collected saliva was all combined in one container (beaker) so that personal information could not be traced. The collected saliva was centrifuged at 2,500×g for 10 minutes, and the supernatant was used as a saliva sample for a solvent in a testing solution.

### 3. Preparation of Testing Solution

### 1) Preparation of Saliva Solution Containing Chewing Gum Components

The amount of the saliva collected when masticating 2 tablets of gum for 5 minutes was 15 ml on average. The sugar alcohol or acidulant was dissolved under stirring in a weight ratio corresponding to 2 tablets of the sample chewing gum into 15 ml of the saliva sample processed with the procedure of 2. That is, to 15 ml of a saliva sample, 2.31 g (15.4%) of the sugar alcohol or 15 mg (0.1%) of the acidulant was added and dissolved. Sodium metaphosphate was dissolved under stirring in an amount needed for each testing. For pH adjustment, an aqueous solution of hydrochloric acid or sodium hydroxide was used.

### 2) Preparation of Extract Solution of Chewing Gum by the Saliva

2 tablets of the chewing gum prewarmed at 50° C., was added 7.5 ml of the saliva sample warmed to 40° C., and extraction was performed by compressing the resultant in a mortar for 5 minutes. This operation was repeated two times and the resultants were combined.

### 4. Preparation of Staining Liquid

For green tea and black tea, 200 ml of distilled water was added to about 20 g of tea leaves and extraction was performed under stirring at 60° C. for 2 hours (green tea; Ito En, Ltd., black tea; Mitsui Norin Co., Ltd.). The extract solution was suction filtered, and the filtrate was freeze-dried to obtain samples. These samples were respectively a green tea extract and a black tea extract.

3% green tea extract, 1% black tea extract, and 1% instant coffee were dissolved under stirring in mineral water at 100° C. to obtain a staining liquid (instant coffee; Ajinomoto General Foods, Inc., mineral water; Suntory Foods Limited).

### 5. Staining and Solution Treatment of Hydroxyapatite Disc

Hydroxyapatite discs (10 mm×10 mm, APP-100, HOYA Corporation) the surfaces of which were uniformly polished with a P400 waterproof abrasive paper were used as human enamel models. The hydroxyapatite discs after polishing were subjected to color measurement by the method of 6. as mentioned below to obtain reference values for the discs. The hydroxyapatite discs were separately put in a 12-well plate (IWAKI&Co., Ltd.) and immersed in 2.5 ml of the saliva sample and allowed to stand still at 37° C. for 2 hours so that saliva pellicles were formed on the surface. The discs were washed with water, and immersed in 2.5 ml of the staining liquid each added in each well of a fresh 12-well plate, and moderately shook at 37° C. for 24 hours so that stains were formed. The hydroxyapatite discs after staining were subjected to color measurement by the method in 6. mentioned below to obtain measurement values after staining. Hydroxyapatite discs after staining subjected to color measurement were placed in a fresh 12-well plate, and 2.5 ml of the saliva sample, the saliva solution containing chewing gum components, or the chewing gum extract solution was dispensed in each well, then the discs were immersed for 5 minutes or 35 minutes. The immersed discs were washed with distilled water, and extra water was wiped off with paper to dry the discs, followed by color measurement.

### 6. Evaluation of Color Difference

A spectrophotometer (CM-700d, Konica Minolta, Inc.) was set upright so that the colorimetric direction was upward. Hydroxyapatite discs before the pellicle formation, after staining or after the solution treatment were allowed to stand still above the colorimetric light source, and color measurements were performed 3 times successively, and the average value was employed. Color measurements were performed on the L*axis (lightness), the a*axis (chroma: red-green axis), and the b*axis (chroma: yellow-blue axis) of the L*a*b*color space based on JIS Z 8722:2009, under the condition of SCI including reflected light. The average value (L*₀, a*₀, b*₀) of the measurement values of the hydroxyapatite discs before staining was taken as the reference value, and using the average value (L*_{after}, a*_{after}, b*_{after}) of the measurement values after staining and after solution treatment, the color difference [symbol] E*ab was calculated according to the following expressions.

ΔE*ab=\sqrt{(ΔL*)²+(Δa*)²+(Δb*)²} [Expression 1]

ΔL*=L*_{after}−L*₀ Δa*=a*_{after}−a*₀ Δb*=b*_{after}−b*₀ [Expression 2]

### 7. Statistical Analysis

The comparison for the transition in the color difference for each group with respect to the repeated testing of stain removal was performed by the two-way analysis of variance with a significant level of 0.05. The comparison between groups per immersion in the chewing gum extract solution was performed by the one-way analysis of variance, followed by Tukey's multiple comparison on data for 7 times (assuming 1 day).

### 8. Results

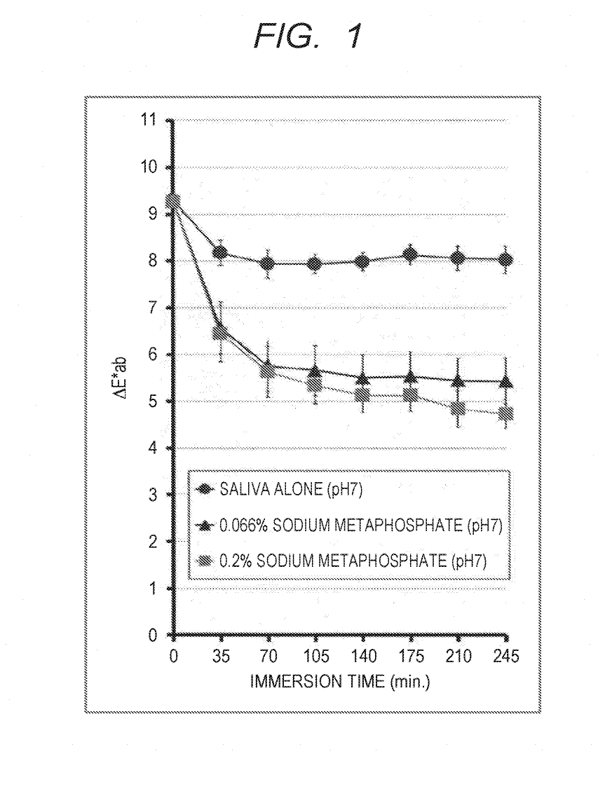

### (1) Confirmation of the Effect of Sodium Metaphosphate in Stain Removal (FIG. 1)

Assuming that when masticating 2 tablets of chewing gum, they are diluted with 15 ml of the saliva, and assuming the mastication of two kinds of chewing gums having the different contents, the stain-removing effects of sodium metaphosphate at a concentration of 0.066% or 0.2% in the saliva were verified. The color differences of all the stained hydroxyapatite discs decreased with the immersion time, and a significant difference was observed compared to the case where the discs were treated with the saliva alone. (FIG. 1, n=6, p<0.05). A significant difference was not observed between the groups of 0.066% and 0.2% sodium metaphosphate.

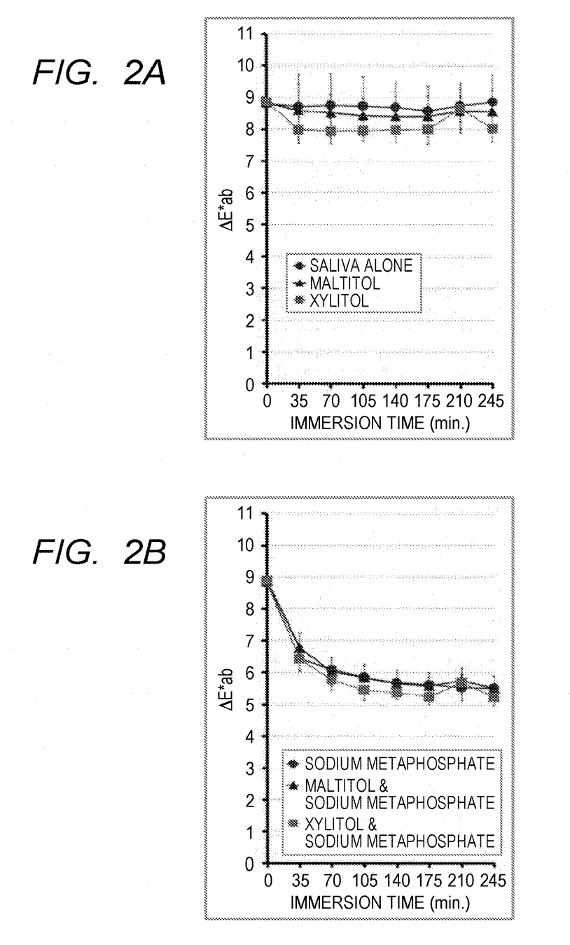

### (2) Influence of Sugar Alcohol on Stain Removal by Sodium Metaphosphate (FIGS. 2A and 2B)

The influence of xylitol and maltitol, sugar alcohols used as sweeteners in chewing gum, on the stain-removing effect by sodium metaphosphate was studied. First, to evaluate whether or not the sugar alcohol itself contributes to stain removal, assuming that all of the sugar alcohol contained in chewing gum is xylitol or maltitol, the stain-removing effect of the saliva solution to which each of the sugar alcohol had been added to give a final concentration of 15.4% was evaluated. Here, a decrease in the color difference of the hydroxyapatite discs was not observed, and a significant difference was not observed when compared to the group with no addition of the sugar alcohol (FIG. 2A, n=6, p<0.05).

Next, each of the sugar alcohol with 0.1% sodium metaphosphate added was prepared, and the influence of the sugar alcohol on the stain-removing effect of sodium metaphosphate was evaluated, then a change in transition of the color difference was not observed (FIG. 2B, n=6, p<0.05).

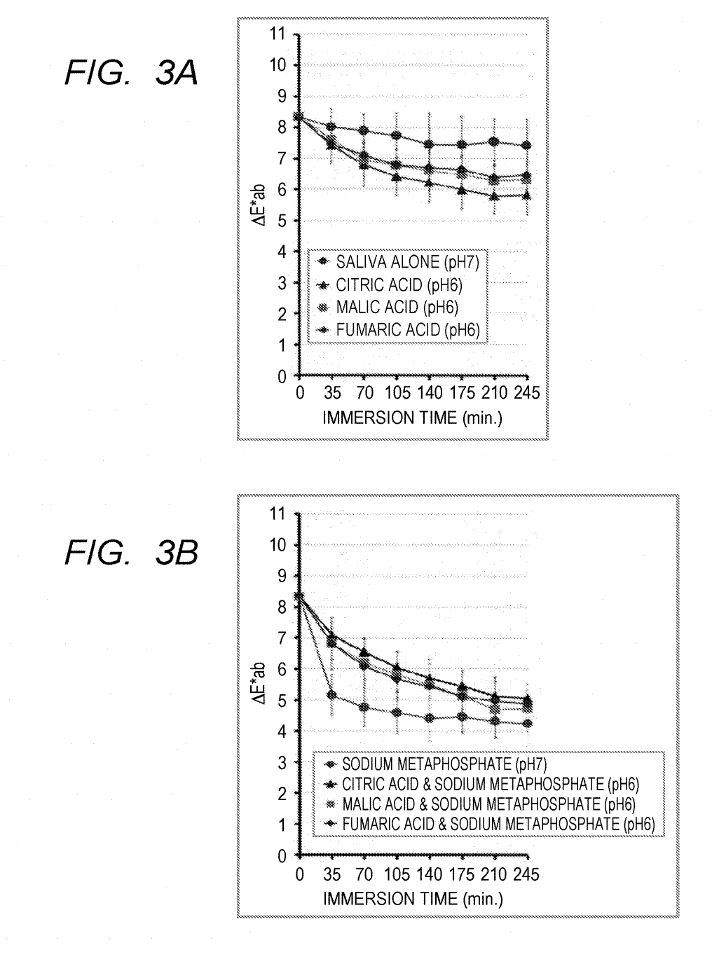

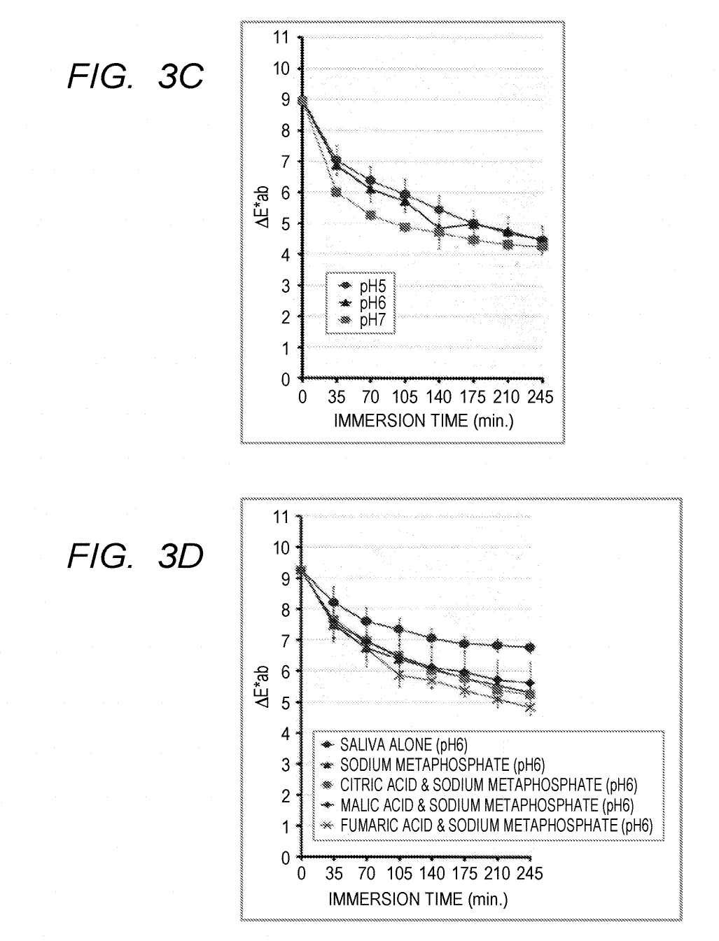

### (3) Influence of Acidulant on Stain Removal by Sodium Metaphosphate (FIGS. 3A to 3D)

Whether or not citric acid, malic acid and fumaric acid used as acidulants in chewing gum themselves contributed to the stain-removing effect was verified. Each acidulant (citric acid, malic acid and fumaric acid) contained in chewing gum was added to the saliva solution so as to be 0.1%. Since the saliva after masticating chewing gum had pH of 6, the saliva solution with the acidulant added was coordinated to be pH 6, and the stain-removing effect was evaluated. As a result, a decrease in the color difference was observed for all the acidulants, and a significant difference was observed compared to the case where the discs had been treated with the saliva alone (pH 7) in the transition of the color difference (FIG. 3A, n=5, p<0.05).

Each of acidulants with 0.1% sodium metaphosphate added thereto was prepared, and whether or not the acidulants had influence on the stain-removing effect of sodium metaphosphate was evaluated. As a result, all the acidulants significantly inhibited a decrease in the color difference compared to the saliva with 0.1% sodium metaphosphate added thereto (pH 7) (FIG. 3B, n=5, p<0.05). Any difference was not observed according to the kinds of the acidulants.

Next, whether or not the inhibitory action of the acidulant on the stain removal of sodium metaphosphate was the influence of pH or the influence of the organic acid anion moiety was investigated.

### 1) Influence of pH on Stain Removal by Sodium Metaphosphate

The saliva with 0.1% sodium metaphosphate added thereto was adjusted to pH 5, 6 and 7 with hydrochloric acid, and the stain-removing effect was evaluated. Then, under the condition of pH 5, a significant difference in the transition of the color difference was observed compared to the case in pH 7. Therefore, it was suggested that protons inhibit the stain-removing effect of sodium metaphosphate (FIG. 3C, n=6, p<0.05).

### 2) Influence of Organic Acid Anion Moiety on Stain Removal by Sodium Metaphosphate

Using the saliva solution to which 0.1% of each acidulant and 0.1% sodium metaphosphate were added at a certain condition of pH 6, the influence of the organic acid anion moiety was evaluated. Any change was not observed in the transition of the color difference (FIG. 3D, n=6, p<0.05).

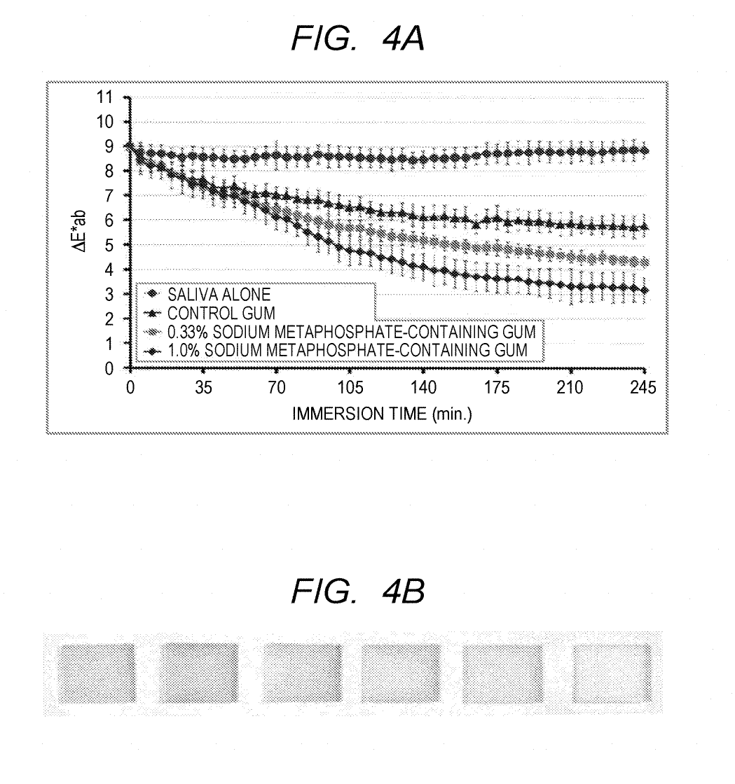

### (4) Stain-Removing Effect of Extract Solution of Sodium Metaphosphate-Containing Chewing Gum (FIGS. 4A and 4B)

Assuming masticating 2 tablets of chewing gum for 5 minutes 7 times daily, and continuing it for 1 week, stained hydroxyapatite discs were immersed in the chewing gum extract solution for 5 minutes at a total of 49 times. Then, the color difference decreased with the times of immersion, and a significant difference was observed in the transition of the color difference when comparing all combinations of the groups (FIG. 4A, n=6, p<0.05). From the multiple comparison per the time of immersion, in the chewing gum extract solution having a final sodium metaphosphate concentration of 0.066% and 0.2% (corresponding to gums containing 0.33% by weight and 1.0% by weight of sodium metaphosphate, respectively) at the sixth time of immersion (treatment for 30 minutes in total), a significant difference was first observed compared to the group treated with the saliva (p<0.05). Moreover, in the discs after 49 times of immersion, stain removal was confirmed visually between the groups (Image 4B). The stain-removing effect was observed dependently on the sodium metaphosphate concentration (0.066%, 0.2%).

When masticating the chewing gum containing sodium metaphosphate aimed at dental stain removal, a sweetener (sugar alcohol) or an acidulant being chewing gum components will be present in the mouth at the same time. The sugar alcohol did not show an inhibitory action on the stain-removing effect of sodium metaphosphate (FIG. 2B), but it was inhibited by the addition of the acidulant (FIG. 3B). It is believed that the cause of this inhibition by the acidulant is not citrate ions, malate ions or fumarate ions, but is a decrease in pH, i.e., an increase in protons (FIGS. 3C and 3D). It is believed that the reason for this is explained by the binding between the tooth and stains. That is, on the dental surface, a pellicle (thin film) is formed by ionic bonding of a negatively charged protein from the saliva to positively charged calcium in hydroxyapatite being the main component of the enamel, and a pigment or the like is deposited on the pellicle to form a stain. It has been reported that, when removing stains with phosphates, the bond between the pellicle and the pigment is not dissociated, but the ionic bond between the dental surface and the pellicle is dissociated by ion exchanges of phosphate ions with the pellicle, so that the stain is removed with the pellicle as one. Based on this surmise, it is believed that the cause of inhibition in the stain-removing effect of sodium metaphosphate by a decrease in pH shown in FIG. 3C in this study is as follows: the ionization equilibrium of metaphosphate moves toward the non-ionized side due to the increase in the hydrogen ion concentration, resulting in a decrease in the metaphosphate ion concentration. That is, it is believed that ion exchanges with the pellicle are reduced due to the decrease in the metaphosphate ion concentration, inhibiting stain removal.

On the other hand, in a stain-removing experiment of the chewing gum extraction solution, a decrease in the color difference was also observed in the extract solution of a control gum. The stain-removing effects of the sugar alcohol and acidulant were checked, then a significant decrease in the color difference was not observed in the sugar alcohol (FIG. 2A). It is believed that this is because the pKas of sugar alcohols are generally high, and they hardly ionize, and similar ion exchanges to that of phosphate ions do not occur. With respect to the addition of the acidulant, considering the facts that a significant decrease in the color difference was observed (FIG. 3A) and that even though citrate ions, malate ions or fumarate ions were added to sodium metaphosphate, more decrease in the color difference was not observed (FIG. 3D), it is believed that the stain-removing effects of organic acid ions are weak, and a main cause of the stain removal by the acidulant is a decrease in pH (decrease in pH from about 7 to 6). It is considered that the reason why citrate ions, malate ions or fumarate ions do not cause a decrease in the color difference in a mechanism of action similar to metaphosphate ions is due to a difference in acid dissociation constants pKas. The pKa of metaphosphoric acid is as small as not measurable in an aqueous system, and it quite easily ionizes compared to citric acid (pKa₁=2.87), malic acid (pKa₁=3.24) and fumaric acid (pKa₁=2.85). That is, it is believed that metaphosphate ions are very stable as anions in solution, and are a substance which easily causes ion exchanges with the pellicle on the dental surface. Therefore, it is suggested that stains deposited every day can be removed by repeated consumption of the chewing gum containing sodium metaphosphate. In general, acidulants are added to chewing gum considering palatability, but because a decrease in pH will suppress the effect of sodium metaphosphate, an even higher effect will be expected in sugarless gum not containing any acidulants.

The stain-removing effect of sodium metaphosphate was not affected by the sugar alcohol, but was inhibited by the addition of the acidulant, and the cause is suggested to be a decrease in pH. In the extract solution in saliva of the sodium metaphosphate-containing chewing gum designed so that the pH in the mouth does not become 6 or lower, a significant stain-removing effect was confirmed. This result suggests that by continuing 7 times daily consumption of the sodium metaphosphate-containing gum for 1 week, a stain-removing effect will be expected. It is noted that in the sodium metaphosphate-containing chewing gum having a stain-removing effect of the invention of the present application, the form of gum may be a stick or block. It also may be tablet gum with a sugar coating, and similar effects will be obtained in either case where sodium metaphosphate is incorporated in the center gum or in the sugar coating.

This application claims priority to Japanese Patent Application No. 2014-011676 filed on Jan. 24, 2014, the contents of which is incorporated herein by reference.

1-6. (canceled)

7. A method for removing stains from teeth, comprising applying to said teeth a stain-removing oral composition containing 0.33% by weight to 2.0% by weight of sodium metaphosphate and 10% by weight to 80% by weight of a sugar alcohol.

8. The stain-removing method according to claim 7, wherein the sugar alcohol is maltitol or xylitol.

9. The stain-removing method according to claim 7, wherein the composition does not cause pH of saliva in the oral cavity to become less than 6.

10. The stain-removing method according to claim 9, wherein the composition causes the pH of saliva in the oral cavity to become about 6.

11. The stain-removing method according to claim 7, wherein the composition further contains 0.5% by weight of an acidulant.

12. The stain-removing method according to claim 11, wherein the acidulant is selected from citric acid, malic acid and fumaric acid.
